(19) United States
(12) Patent Application Publication
(10) Pub. No.: US 2020/0345762 A1
(43) Pub. Date: Nov. 5, 2020

(54) METHOD AND SYSTEM FOR PROVIDING PERITONEAL DIALYSIS FLUIDS WITH VARYING SODIUM CONCENTRATIONS

(71) Applicant: GAMBRO LUNDIA AB, Lund (SE)

(72) Inventors: Olof JANSSON, Vellinge (SE); Anders WIESLANDER, LUND (SE)

(21) Appl. No.: 16/760,570

(22) PCT Filed: Oct. 25, 2018
(86) PCT No.: PCT/EP2018/079245

§ 371 Date: Apr. 30, 2020

Related U.S. Application Data
(60) Provisional application No. 62/581,310, filed on Nov. 3, 2017.

(51) Int. Cl. A61K 33/06; A61K 31/7004; A61K 33/00; A61K 47/02; A61K 47/12; A61K 9/00; A61M 1/28

(52) U.S. Cl. CPC A61K 33/06; A61K 31/7004; A61K 33/00; A61M 1/287; A61K 47/12; A61K 9/0026; A61K 47/02

(57) ABSTRACT

Methods are provided for preparing a ready-to-use peritoneal dialysis fluid for peritoneal dialysis of a patient. The methods comprise mixing, immediately before administration to the patient, appropriate amounts of at least a first concentrate and a second concentrate with an appropriate amount of water to form a ready-to-use dialysis fluid. The first concentrate comprises glucose and has a pH of between 1.5 and 4; and the second concentrate comprises a physiologically acceptable buffer and sodium ions, and has a pH of between 5.0 and 9.0. The ready-to-use dialysis fluid has a sodium ion concentration of about 110 mM to about 145 mM and an osmolality of about 0.20 to about 0.50 Osm/kg.

### BACKGROUND

### Field

The disclosure relates to the field of fluid compounding for preparing fluids particularly for the treatment of renal insufficiency. More specifically, it relates to a method for compounding finished fluids from two or more concentrates for use as a dialysis fluid for the treatment of renal insufficiency. In particular, the methods may be used for preparing fluids for peritoneal dialysis, particularly for preparing fluids at the point-of-care of the patient.

### Description of the Related Art

Patients with acute or chronic renal insufficiency may need supporting treatment in the form of dialysis for the removal of waste substances and excess of fluid from the body. Dialysis is a process to remove fluid and waste products from the patient by the use of diffusive or convective transport. Various dialysis techniques with associated dialysis fluids may be differentiated. Which dialysis technique to use depends on the patient needs, treatment demands and available resources.

Peritoneal dialysis is one available dialysis technique for patients having renal failure. During this treatment, a peritoneal dialysis fluid is infused in the peritoneal cavity of the patient via a catheter inserted through the abdominal wall. In peritoneal dialysis, the peritoneal membrane serves as the dialysis membrane. An osmotic pressure gradient is applied by the addition of an osmotic agent to the dialysis fluid which will cause fluid removal from the blood. The amount of fluid removed during the dialysis treatment depends on the concentration of the osmotic agent chosen in the fluids used; the higher concentration, the larger amount of fluid is removed. Methods of peritoneal dialysis treatment include, for example, Continuous Ambulatory Peritoneal dialysis (CAPD), Continuous Flow Peritoneal Dialysis (CFPD), Intermittent Peritoneal Dialysis (IPD), Tidal Peritoneal Dialysis (TPD) and Automated Peritoneal Dialysis (APD).

In automated peritoneal dialysis, an automated cycler is used to infuse and drain dialysis fluid. This form of treatment may be done automatically at night while the patient sleeps. The cycler measures the amount of fluid infused and the amount removed to compute the net fluid removal. The treatment sequence usually begins with an initial drain cycle to empty the peritoneal cavity of dialysate (also called spent dialysis fluid). The cycler then performs a series of fill, dwell, and drain cycles, typically finishing with a fill cycle.

Peritoneal dialysis generally requires large volumes of dialysis fluids. Generally, at each application, or exchange, a given patient will infuse 0.5 to 3 liters of dialysis fluid into the peritoneal cavity. The fluid is allowed to dwell for approximately 1-4 hours, at which time it is drained out and exchanged for fresh fluid. Generally, four such exchanges are performed daily. Approximately 8 to 20 liters of dialysis fluid is required per day, 7 days a week, 365 days a year for each patient.

The peritoneal dialysis fluids have traditionally been provided in bags, often as 1.5 L, 2 L, 3 L, 5 L, or 6 L bags, and being terminally sterilized. Shipping and storage of the sheer volume of fluids required is both tremendously inconvenient and expensive. Further, for the patient, the repeated connection and disconnection of multiple fluid containers creates a risk of microbiological contamination at the point of connection. Additionally, large amounts of waste material, in form of empty containers and packaging, and their proper disposal are increasingly becoming a concern.

Further, patients with low residual renal function may become fluid overloaded. The problem typically increases with time on dialysis treatment, not only due to loss of residual renal function, but also due to changes in the peritoneal membrane. Conventional approaches to manage fluid overload include dietary salt and fluid restriction, use of diuretics, anti-hypertensive drugs, icodextrin based dialysis solutions, addition of an extra day dwell, or switching to hemodialysis.

A need therefore exists for an improved method and system for delivering varying sodium concentrations to peritoneal dialysis patients.

### SUMMARY

The disclosure provides a method for preparing a ready-to-use peritoneal dialysis fluid for treating a dialysis patient. The methods have flexibility and capability to provide ready-to-use peritoneal dialysis fluids having various sodium concentrations. For example, the methods provide ready-to-use peritoneal dialysis fluids having sodium concentrations within a desired range, including standard concentrations such as, for example, 132 mM, and concentrations above or below such standard concentration (i.e., concentrations above or below 132 mM). Advantageously, such low sodium dialysis fluids increase sodium removal by diffusion, thereby facilitating management of fluid overload during long term use. Further, the methods disclosed herein avoid the problems of shipping and storing large volumes of ready-to-use dialysis fluids. In particular, the disclosure provides methods wherein small volumes of concentrated dialysis fluids are combined and diluted with purified water at the point of care, i.e. close to the patient. A first concentrate comprises glucose, allowing different volumes of glucose to be dosed to obtain different glucose concentrations. A second concentrate comprises a physiologically acceptable buffer and sodium ions, allowing different volumes of sodium to be dosed to obtain different sodium concentrations.

In one embodiment, which may be combined with any of the embodiments disclosed herein unless specified otherwise, the method comprises mixing, immediately before administration to the patient, appropriate amounts of at least a first concentrate and a second concentrate with an appropriate amount of water to form a ready-to-use dialysis fluid; wherein the first concentrate comprises glucose, has a pH of between 1.5 and 4, low levels of glucose degradation products and is free of sodium ions; the second concentrate comprises a physiologically acceptable buffer and sodium ions, and has a pH of between 5.0 and 9.0; and the ready-to-use dialysis fluid has a sodium ion concentration of about 110 mM to about 145 mM and an osmolality of about 0.20 to about 0.50 Osm/kg, for example, 0.3 Osm/kg.

In one embodiment, which may be combined with any of the embodiments disclosed herein unless specified otherwise, the ready-to-use dialysis fluid has a sodium ion concentration of about 110 mM to about 132 mM, such as about 115 mM to about 125 mM.

In one embodiment, which may be combined with any of the embodiments disclosed herein unless specified otherwise, the ready-to-use dialysis fluid has a sodium ion concentration of about 120 mM to about 145 mM, such as about 132 mM to about 145 mM.

In one embodiment, which may be combined with any of the embodiments disclosed herein unless specified otherwise, the physiologically acceptable buffer is selected from the group consisting of acetate, lactate, citrate, pyruvate, carbonate, bicarbonate, amino acid buffers, and mixtures thereof. For example, the physiologically acceptable buffer comprises lactate, bicarbonate, or a mixture thereof.

In one embodiment, which may be combined with any of the embodiments disclosed herein unless specified otherwise, the second concentrate further comprises at least one electrolyte selected from the group consisting of calcium, magnesium, and potassium.

In one embodiment, which may be combined with any of the embodiments disclosed herein unless specified otherwise, the first concentrate and/or the second concentrate is configured to be used for dilutions of between 1:10 and 1:50.

In one embodiment, which may be combined with any of the embodiments disclosed herein unless specified otherwise, the water is sterile water produced by reverse osmosis and sterile filtration.

In one embodiment, which may be combined with any of the embodiments disclosed herein unless specified otherwise, the ready-to-use dialysis fluid contains: 110-145 mM sodium (Na⁺), 0-4 mM potassium (K⁺), 0-2 mM calcium (Ca²⁺), 0-0.75 mM magnesium (Mg²⁺), 0-50 mM lactate, 0-50 mM bicarbonate, and 0-5% glucose.

In one embodiment, which may be combined with any of the embodiments disclosed herein unless specified otherwise, the ready-to-use dialysis fluid contains: 110-145 mM sodium (Na⁺), 0-4 mM potassium (K⁺), 1.25-1.75 mM calcium (Ca²⁺), 0-0.75 mM magnesium (Mg²⁺), 35-50 mM lactate, and 0-5% glucose.

In one embodiment, which may be combined with any of the embodiments disclosed herein unless specified otherwise, the first concentrate further comprises an acid selected from HCl and organic acids.

In one embodiment, which may be combined with any of the embodiments disclosed herein unless specified otherwise, the ready-to-use dialysis fluid is used for automated peritoneal dialysis (APD).

In one embodiment, which may be combined with any of the embodiments disclosed herein unless specified otherwise, the first concentrate comprises 25-60% glucose.

In one embodiment, which may be combined with any of the embodiments disclosed herein unless specified otherwise, the second concentrate comprises: 1-5.5 M sodium (Na⁺), 0-0.15 M calcium (Ca²⁺), 0-0.03 M magnesium (Mg²⁺), 0-0.1 M potassium (K⁺), 0-1.6 M lactate, and 0-1.6 M bicarbonate.

In one embodiment, which may be combined with any of the embodiments disclosed herein unless specified otherwise, the ready-to-use dialysis fluid is prepared using a system comprising: a) a proportioning device or peritoneal dialysis cycler operating a disposable pump and valve set; b) at least one source of water adapted for connection with said disposable pump and valve set operated by the proportioning device; c) at least one source of the first concentrate adapted for connection with said disposable pump and valve set; and d) at least one source of the second concentrate adapted for connection with said disposable pump and valve set. Of course, the principle of the invention can also be put into practice with a different machine and process, as long as the ready-to-use solution is generated from at least one source of the first concentrate, at least one source of the second concentrate and at least one source of the third concentrate.

In one embodiment, which may be combined with any of the embodiments disclosed herein unless specified otherwise, each concentrate may be dispensed from multiple canisters or bags.

In one embodiment, which may be combined with any of the embodiments disclosed herein unless specified otherwise, a proportioning device includes a controller programmed to cause the proportioning device to perform multiple fills using the ready-to-use dialysis fluid as a patient fill solution, wherein (i) the fill solutions of each patient fill contain the second concentrate, (ii) the fill solutions of less than all patient fills contain the second concentrate, (iii) the fill solutions of each patient fill contain a like, or substantially like, amount or concentration of the second concentrate, and/or (iv) one or more or all of the fill solutions of the multiple patient fills contain a different amount or concentration of the second concentrate.

In one embodiment, which may be combined with any of the embodiments disclosed herein unless specified otherwise, a method is provided for preparing a ready-to-use peritoneal dialysis fluid for peritoneal dialysis of a patient. The method includes mixing, immediately before administration to a patient, appropriate amounts of at least a first concentrate free from sodium ions and a second concentrate comprising sodium ions with an appropriate amount of water to form a ready-to-use dialysis fluid.

In one embodiment, which may be combined with any of the embodiments disclosed herein unless specified otherwise, the ready-to-use dialysis fluid has a sodium ion concentration of about 110 mM to about 132 mM.

In one embodiment, which may be combined with any of the embodiments disclosed herein unless specified otherwise, a system is provided for preparing a ready-to-use peritoneal dialysis fluid for peritoneal dialysis of a patient. The system may include a proportioning device or cycler; at least one source of water adapted for connection with the proportioning device or cycler; at least one source of the first concentrate free from sodium ions adapted for connection with the device or cycler and the source of water; and at least one source of the second concentrate adapted for connection with the device or cycler and the source of water.

### BRIEF DESCRIPTION OF THE DRAWINGS

FIG. 1 provides scheme 100 showing a system for preparing a ready to use peritoneal dialysis fluid. A container 10 comprising a first concentrate and a container 12 comprising a second concentrate are each connected to a proportioning device 16 or cycler for mixing by conduits. A source 14 of purified water is also connected to the mixer, which may be part of a disposable pump and valve set operated by proportioning device or cycler 16, by a conduit. The proportioning device or cycler 16 is controlled by a controller 18 based on input from a user interface 20. Depending on such user input and control signals from the controller 18, the proportioning device 16 receives specific amounts of first and second concentrates, as well as water, from said sources 10, 12, and 14, and produces a ready-to-use peritoneal dialysis fluid that is delivered through output/container 22.

### DETAILED DESCRIPTION

### Definitions

The term “first concentrate” means herein the source of glucose. The source may be provided as fluid concentrate.

The term “second concentrate” means herein the source of physiologically acceptable buffer and of sodium. Examples of physiologically acceptable buffers are acetate, lactate, citrate, pyruvate, carbonate, bicarbonate, and amino acid buffer, if not otherwise specified. Further, the buffers are intended to be in form of alkali, for example alkali lactate, and alkali bicarbonate, such as sodium lactate, and sodium bicarbonate.

The term “lactate” means lactic acid or any salt thereof. For example, the salt may be formed with sodium, potassium, calcium, or magnesium.

The term “terminal sterilized” is herein intended to mean that the product is sterilized in its final package. The terminal sterilization may include heat sterilization and/or radiation sterilization, but is preferably heat sterilization effected in an autoclave at a temperature of at least 100° C., preferably at least 121° C.

The term “dilution” as used herein refers to the mixing of a small, measured sample with a large volume of, for example, sterile water, saline or other appropriate liquid called the diluent or a dilution blank. A single dilution is calculated as follows:

Dilution=volume of the sample/(sum of the volume of the sample and the diluent volume).

For example, the dilution of 1 mL into 9 mL equals: 1/(1+9), which is the same as 1/10 which is written 1/10 or 10⁻¹. This can then be called a one to ten dilution.

### Peritoneal Dialysis Fluids

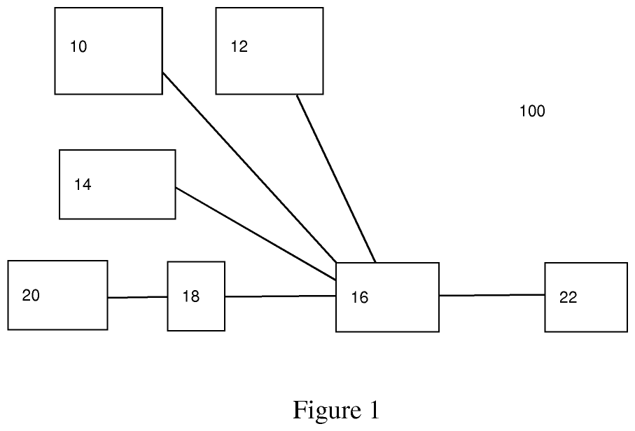

The disclosure provides methods for preparing a ready-to-use peritoneal dialysis fluid for peritoneal dialysis of a patient. The methods comprise: mixing using the cycler 16 illustrated in FIG. 1, before administration to the patient, appropriate amounts of at least a first concentrate and a second concentrate with an appropriate amount of water to form a ready-to-use dialysis fluid. In an embodiment, the ready-to-use peritoneal dialysis fluid may be used for automated peritoneal dialysis (APD). The method may comprise mixing the concentrates with water immediately prior to administration to the patient.

In the disclosed methods, the first concentrate comprises glucose and is free of sodium ions. The first concentrate has a pH of between 1.5 and 4. For example, the pH is between 1.5 and 3, between 2 and 3.5, between 2.2 and 3.2, between 2.2 and 3, between 2.2 and 2.8, or between 2.4 and 2.8. The concentrate is acidified by, for example, addition of hydrochloric acid (HCl) or an organic acid. Examples of organic acids are citric acid and acetic acid. The concentrate may comprise 25-60% glucose, for example 25-40%, 30-50%, or 40-60%. Advantageously, using a concentrate comprising glucose provides great flexibility in the concentration of glucose obtained for the ready-to-use fluid. The first concentrate is free of sodium ions, which provides greater flexibility to adjust the glucose concentration of the ready-to-use fluid without affecting the sodium concentration. This is particularly advantageous given the limits provided in the European Pharmacopoeia on the amount of sodium ions in a ready-to-use peritoneal dialysis fluid (±2.5% in relation to the target amount).

In the disclosed methods, the second concentrate comprises a physiologically acceptable buffer and sodium ions. Suitable buffers include, but are not limited to, acetate, lactate, citrate, pyruvate, carbonate, bicarbonate, amino acid buffers (e.g., histidine), and mixtures thereof. For example, the buffer comprises lactate, bicarbonate, or a mixture thereof. The second concentrate typically has a pH of between 5.0 and 9, for example between 5.0 and 8.5, between 6.5 and 9, between 6 and 8.5, between 6.5 and 8.5, or between 6.8 and 8.5. The second concentrate optionally comprises one or more additional electrolytes. Suitable electrolytes include, but are not limited to, calcium, and magnesium, and potassium. A second concentrate as disclosed herein may have the following content:

| | |
|---|---|
| sodium (Na⁺) | 1-5.5M, for example, 2-4M, 2-3M, or 3-4M |
| calcium (Ca²⁺) | 0-0.15M, for example, 0-0.12M, or 0.05-0.1M |
| magnesium (Mg²⁺) | 0-0.03M, for example, 0.01-0.02M |
| potassium (K⁺) | 0-0.1M |
| lactate | 0-1.6M, for example, 0.5-1M |
| bicarbonate | 0-1.6M, for example, 0.5-1M |

Using the first and second concentrates as disclosed herein advantageously provides methods wherein great flexibility in the concentration of sodium is achieved. Because the concentrations of other electrolytes and buffers in the second concentrate are much lower relative to the sodium concentration, the therapeutic effect of fluctuations in the concentrations of these other electrolytes and buffers will be marginal compared to the therapeutic effect of the sodium concentration. In an embodiment, the methods disclosed herein enhance sodium removal during peritoneal dialysis compared to conventional peritoneal dialysis methods by providing ready-to-use peritoneal dialysis fluids having a desired range or concentration, including standard concentrations such as, for example, 132 mM, and concentrations above or below such standard concentrations (i.e., concentrations below or above 132 mM). Without wishing to be bound by theory, it is believed that while increasing the glucose concentration of a peritoneal dialysis fluid to obtain more ultrafiltration will also increase absolute sodium removal, the relative proportion of sodium to fluid removal will hence decrease, especially during short exchanges, due to aquaporin-mediated fluid transport. The ability to, for example, lower the sodium concentration in the peritoneal dialysis fluid in concurrence with an increased glucose concentration will facilitate increased sodium removal by diffusion, thereby reducing the gap between sodium and water losses. In some cases, low sodium concentrations, which decrease the osmolality of the ready-to-use dialysis fluids, result in undesirably low levels of ultrafiltration. In an embodiment, the glucose concentration of the low-sodium ready-to-use dialysis fluid is increased to compensate for the reduced sodium osmolality, thereby maintaining a desirable level of ultrafiltration and providing a ready-to-use dialysis fluid suitable for treatment of fluid overload.

The first concentrate and the second concentrate may be configured to be used for dilutions of between 1:10 and 1:50 to obtain the ready-to-use peritoneal dialysis solution. For example, the first or second concentrate may be configured to be used for dilutions of between 1:12 and 1:38, between 1:13 and 1:37, between 1:15 and 1:35, between 1:20 and 1:30, or between 1:25 and 1:30, based on the total volume of the ready-to-use dialysis fluid. The level of concentrate also is referred to as 10×, 15×, 20×, 25×, 30×, 35×, and 50×. The concentrates of components for preparing the ready-to-use peritoneal dialysis fluid may each be provided in volumes of about 0.5 L to about 10 L, for example, about 1 L to about 3 L or about 1 L to about 2 L, or about 5 L. Advantageously, these concentrates having smaller volumes will replace the 8-55 L of peritoneal dialysis fluid typically used by patients.

The pH of the ready-to-use peritoneal dialysis fluids disclosed herein typically is between 5-8, for example between 6.5-7.5, or between 6.8-7.5, or between 6.0-8.5. The ready-to-use peritoneal dialysis fluids typically have a pH close to physiological/neutral to reduce infusion pain.

The sodium ion concentration of the ready-to-use peritoneal dialysis fluids disclosed herein is about 90 to about 145 mM, for example, about 110 to about 132 mM, about 115 to about 125 mM, about 120 to about 130 mM, about 120 to about 125 mM, about 125 to about 135 mM, about 130 to 135 mM, about 132 to about 145 mM, about 135 to about 145 mM, about 140 to about 145 mM, or about 132 mM.

The potassium ion concentration of the ready-to-use peritoneal dialysis fluids disclosed herein typically is 0 to about 4 mM, for example, about 0.5 to about 4 mM, about 1 to about 4 mM, about 1.6 to about 4 mM, about 1.6 to about 3 mM, or about 1.6 to about 2 mM.

The lactate concentration of the ready-to-use peritoneal dialysis fluids disclosed herein typically is about 0 mM to about 50 mM, about 10 mM to about 40 mM, about 15 mM to about 40 mM, about 20 mM to about 50 mM, about 30 mM to about 50 mM, or about 35 mM to about 40 mM.

A ready-to-use peritoneal dialysis solution as disclosed herein has an osmolality of about 0.20 to about 0.50 Osm/kg, for example, about 0.28 to about 0.49 Osm/kg, or about 0.29 to about 0.3 Osm/kg.

A ready-to-use peritoneal dialysis solution as disclosed herein may have the following content:

| | |
|---|---|
| sodium (Na⁺) | 110-145 mM, for example, 115-125 mM, 110-132 mM, 110-130 mM, |
| | 110-125 mM, 125-135 mM, 132-145 mM, 130-135 mM, 132 mM, or |
| | 140-145 mM |
| potassium (K⁺) | 0-4 mM, for example, 0.5-4 mM, 1-4 mM, 1.6-4 mM, 1.6-3 mM, or |
| | 1.6-2 mM |
| calcium (Ca²⁺) | 0-2 mM, for example, 0.5-2 mM, 1-2 mM, 1-1.75 mM, or 1.25-1.5 |
| | mM |
| magnesium (Mg²⁺) | 0-0.75 mM, for example, 0.1-0.75 mM, 0.25-0.75 mM, 0.25-0.5 mM, |
| | 0.3-0.45 mM, or 0.35-0.4 |
| lactate | 0-50 mM, for example, 10-50 mM, 15-40 mM, 20-40 mM, 30-40 mM, |
| | or 35-40 mM |
| bicarbonate | 0-50 mM, for example, 10-45 mM, 15-40 mM, 20-40 mM, 25-40 mM, |
| | 30-40 mM, or 35-40 mM |
| glucose | 0-5%, for example, 0.5-5%, 1-5%, 1.3-1.5%, 1.5-5%, 1.5-4.25%, 2- |
| | 5%, 2.5-5%, 3-5%, 3.8-4.0%, or 4.25-5% |

A ready-to-use peritoneal dialysis solution as disclosed herein may have the following content:

| | | |
|---|---|---|
| sodium (Na⁺) | 110-145 | mM |
| potassium (K⁺) | 0-4 | mM |
| calcium (Ca²⁺) | 0-2 | mM |
| magnesium (Mg²⁺) | 0-0.75 | mM |
| lactate | 0-50 | mM |
| bicarbonate | 0-45 | mM |

| | |
|---|---|
| glucose | 0-5% |

A ready-to-use peritoneal dialysis solution as disclosed herein may have the following content:

| | |
|---|---|
| sodium (Na⁺) | 90-145 mM, for example, 110-132 mM, 115-125 mM, 120-130 mM, |
| | 120-125 mM, 125-135 mM, 132-145 mM, 135-145 mM, or 140-145 |
| | mM |
| potassium (K⁺) | 0-4 mM, for example, 0.5-4 mM, 1-4 mM, 1.6-4 mM, 1.6-3 mM, or |
| | 1.6-2 mM |
| calcium (Ca²⁺) | 1.25-1.75 mM, for example, 1.5-1.75 mM or 1.25-1.5 mM |
| magnesium (Mg²⁺) | 0-0.75 mM, for example, 0.1-0.75 mM, 0.25-0.75 mM, 0.25-0.5 mM, |
| | 0.3-0.45 mM, or 0.35-0.4 |
| lactate | 35-40 mM |
| glucose | 0-5%, for example, 1.3-4.25%, 2-5%, 2.5-5%, 3-5%, or 4.25-5% |

A ready-to-use peritoneal dialysis solution as disclosed herein may have the following content:

| | | |
|---|---|---|
| sodium (Na⁺) | 90-145 | mM |
| potassium (K⁺) | 0-4 | mM |
| calcium (Ca²⁺) | 1.25-1.75 | mM |
| magnesium (Mg²⁺) | 0-0.75 | mM |
| lactate | 35-40 | mM |

| | |
|---|---|
| glucose | 0-5% |

The list of examples of ready-to-use peritoneal dialysis fluids is not exhaustive or intended to limit the present invention.

The system for preparing a ready-to-use peritoneal dialysis solution as disclosed herein may comprise the following: a) a proportioning device or cycler operating a disposable valve and pump set; b) at least one source of water adapted for connection with said disposable valve and pump set operated by the proportioning device or cycler; c) at least one source of the first concentrate adapted for connection with a) and b); and d) at least one source of the second concentrate adapted for connection with a) and b).

The system as is described herein comprises a proportioning device or cycler. In the proportioning device are the concentrates admixed, i.e. proportioned and compounded, to form a ready-to-use peritoneal dialysis fluid. With the system as defined herein there is provided a ready way to prepare the dialysis fluid for the peritoneal dialysis treatment. Less amount and less volumes of concentrates are to be handled in connection with the treatment of the patient.

Systems for peritoneal dialysis and/or proportioning devices also are described in International Application Nos. PCT/US2017/031396, PCT/EP2017/060769, WO 2013/1141896, WO 2012/129501, U.S. application Ser. Nos. 15/588,220, 15/588,235, 15/588,454, and U.S. Pat. No. 5,344,392 which are incorporated herein by reference in their entireties. Commercially available devices or cyclers may be used for proportioning including, for example, AMIA® APD Machine (Baxter International Inc).

The system for preparing a ready-to-use peritoneal dialysis solution as disclosed herein includes at least one water source. The water to be mixed with the concentrate(s) included in this production shall have a certain chemical and microbiological quality (defined in e.g. European Pharmacopoeia) suitable for its application.

Water to be included in the source of water should be within limits that are safe from a microbiological and chemical perspective; this water could for example be “purified water”, “highly purified water”, “ultrapure water”, “water for injection” (WFI), “sterile WFI”, “water for hemodialysis”, “distilled water”, “sterile purified water” and “water for pharmaceutical use”. For example, the water to be included in the source of water may be sterile water produced by reverse osmosis and/or sterile filtration.

The herein defined first concentrate and second concentrate may be terminal sterilized before they are included in the system. By having sterilized concentrate, by for example terminal sterilization, included in the system these can be mixed with the water having the quality as defined above and a ready-to-use peritoneal dialysis fluid of high quality is provided. There is no requirement of sterilization of the ready-to-use peritoneal dialysis fluid. By the invention, it is possible to provide the ready-to-use peritoneal dialysis fluid close to the point of care.

Alternatively, the first and second concentrate may not be sterilized before inclusion in the system and may be mixed with water purified by reverse-osmosis. The concentrates and water may then be sterile filtered.

FIG. 1 illustrates a system 100 showing a system for preparing a ready-to-use peritoneal dialysis fluid. A container 10 comprising a first concentrate and a container 12 comprising a second concentrate are each connected to a disposable pump and valve set operated by a proportioning device or cycler 16 for mixing by conduits. A source 14 of purified water is also connected to the disposable pump and valve set operated by the proportioning device or cycler 16 by a conduit. The proportioning device or cycler 16 is controlled by a controller 18 based on input from a user interface 20. Depending on such user input and control signals from the controller 18, the proportioning device 16 receives specific amounts of first and second concentrates, as well as water, from sources 10, 12, and 14, and produces a ready-to-use peritoneal dialysis fluid that is delivered through output/container 22.

Proportioning device or cycler 16 under control of controller 18 is in various embodiments configured or programmed to perform multiple drain, fill and dwell cycles (if the patient is initially full with a previous treatment's last fill or midday exchange) or multiple fill, dwell and drain cycles (if the patient starts treatment empty). In either case, it is contemplated to infuse any of the sodium containing concentrates described herein according to a device prescription prepared by a doctor or clinician, wherein: (i) all patient fill solutions contain sodium, (ii) less than all patient fill solutions contain sodium, (iii) each patient fill solution containing sodium contains a like, or substantially like, amount or concentration of sodium, and/or (iv) one or more or all of the patient fill solutions containing sodium contain a different amount or concentration of sodium.

In some embodiments, the second concentrate may comprise the following composition:
11.34 g/100 ml NaCl (Mw=58.44 g/mol); 1940 mM
7.84 g/100 ml sodium lactate (Mw=112.06 g/mol); 700 mM
514 mg/100 ml calcium chloride (Mw (CaCl₂)*2H₂O)=147.01 g/mol); 35.0 mM
304 mg/100 ml magnesium chloride (Mw (hexahydrate)=203.31 g/mol); 14.95 mM, with a pH of approximately 6.4.
   In other embodiments, the second concentrate may not comprise the above composition.

While the invention has been described in connection with what is presently considered to be the most practical embodiments, it is to be understood that the invention is not to be limited to the disclosed embodiments, but on the contrary, is intended to cover various modifications and equivalents included within the spirit and the scope of the appended claims.

1-21. (canceled)

22. A method for preparing a ready-to-use peritoneal dialysis fluid for peritoneal dialysis of a patient, the method comprising:
mixing, before administration to the patient, appropriate amounts of at least a first concentrate and a second concentrate with an appropriate amount of water to form a ready-to-use dialysis fluid,
wherein the first concentrate includes glucose, has a pH of between 1.5 and 4, and is free of sodium ions,
wherein the second concentrate includes a physiologically acceptable buffer and sodium ions, and has a pH of between 5.0 and 9.0, and
wherein the ready-to-use dialysis fluid has a sodium ion concentration of about 110 mM to about 145 mM and an osmolality of about 0.20 to about 0.5 Osm/kg.

23. The method of claim 22, wherein the ready-to-use dialysis fluid has a sodium ion concentration of about 110 mM to about 132 mM.

24. The method of claim 22, wherein the ready-to-use dialysis fluid has a sodium ion concentration of about 115 mM to about 125 mM.

25. The method of claim 22, wherein the ready-to-use dialysis fluid has a sodium ion concentration of about 130 mM to about 135 mM.

26. The method of claim 22, wherein the ready-to-use dialysis fluid has a sodium ion concentration of about 135 mM to about 145 mM.

27. The method of claim 22, wherein the physiologically acceptable buffer includes one or more selected from the group consisting of: acetate, lactate, citrate, pyruvate, carbonate, bicarbonate and amino acid buffers.

28. The method of claim 27, wherein the physiologically acceptable buffer includes one or both of lactate and bicarbonate.

29. The method of claim 22, wherein the second concentrate further includes at least one electrolyte selected from the group consisting of: calcium, magnesium and potassium.

30. The method of claim 22, wherein the first concentrate or the second concentrate is adapted to be used for dilutions of between 1:10 and 1:50.

31. The method of claim 22, wherein the water is sterile water produced by reverse osmosis or distillation and sterile filtration.

32. The method of claim 22, wherein the ready-to-use dialysis fluid includes:
90 to 145 mM sodium (Na⁺),
0 to 4 mM potassium (K⁺),
0 to 2 mM calcium (Ca²⁺),
0 to 0.75 mM magnesium (Mg²⁺),
0 to 50 mM lactate,
0 to 50 mM bicarbonate, and
0 to 5% glucose.

33. The method of claim 22, wherein the ready-to-use dialysis fluid includes:
90 to 145 mM sodium (Na⁺),
0 to 4 mM potassium (K⁺),
1.25 to 1.75 mM calcium (Ca²⁺),
0 to 0.75 mM magnesium (Mg²⁺),
35 to 40 mM lactate, and
0 to 5% glucose.

34. The method of claim 22, wherein the first concentrate further includes an acid selected from HCl and organic acids.

35. The method of claim 22, wherein the ready-to-use dialysis fluid is adapted for automated peritoneal dialysis.

36. The method of claim 22, wherein the first concentrate includes 25 to 60% glucose.

37. The method of claim 22, wherein the second concentrate includes:
1.0 to 5.5 M sodium (Na⁺),
0 to 0.15 M calcium (Ca²⁺),
0 to 0.03 M magnesium (Mg²⁺),
0 to 1.6 M lactate, and
0 to 1.6 M bicarbonate.

38. The method of claim 22, wherein the ready-to-use dialysis fluid is prepared using a system including:
a proportioning device;
at least one source of water adapted for connection with the proportioning device;
at least one source of the first concentrate adapted for connection with the proportioning device and the at least one source of water; and
at least one source of the second concentrate adapted for connection with the proportioning device and the at least one source of water.

39. The method of claim 38, wherein the proportioning device includes a controller programmed to cause the proportioning device to perform multiple fills using the ready-to-use dialysis fluid as a patient fill solution, wherein the controller is programmed to effect at least one from the group consisting of: (i) the patient fill solutions of each fill contain the second concentrate, (ii) the patient fill solutions of less than all fills contain the second concentrate, (iii) the patient fill solutions of each fill contain a like, or substantially like, amount or concentration of the second concentrate, and (iv) one or more or all of the patient fill solutions of the multiple fills contain a different amount or concentration of the second concentrate.

40. The method of claim 22, wherein the mixing occurs immediately before administration to the patient.

41. A method for preparing a ready-to-use peritoneal dialysis fluid for peritoneal dialysis of a patient, the method comprising:
mixing, before administration to the patient, appropriate amounts of at least a first concentrate and a second concentrate with an appropriate amount of water to form a ready-to-use dialysis fluid,
wherein the first concentrate is free of sodium ions, and
wherein the second concentrate includes sodium ions.

42. The method of claim 41, wherein the ready-to-use peritoneal dialysis fluid has a sodium ion concentration of about 110 mM to about 132 mM.

43. The method of claim 41, wherein the mixing occurs immediately before administration to the patient.

44. A system for preparing a ready-to-use peritoneal dialysis fluid for peritoneal dialysis of a patient, the system comprising:
a proportioning device or cycler;
at least one source of water adapted for connection with the proportioning device or cycler;
at least one source of the first concentrate adapted for connection with (i) the proportioning device or cycler and (ii) the source of water, wherein the first concentrate is free from sodium ions; and
at least one source of the second concentrate adapted for connection with (i) the proportioning device or cycler and (ii) the source of water.
